(19) United States
(12) Patent Application Publication
(10) Pub. No.: US 2017/0258487 A1
(43) Pub. Date: Sep. 14, 2017

(54) SURGICAL TREATMENT INSTRUMENT AND SURGICAL TREATMENT INSTRUMENT APPARATUS

(71) Applicant: OLYMPUS CORPORATION, Tokyo (JP)

(72) Inventors: Hikaru JINBO, Saitama-shi (JP); Hiroshi ITO, Tokyo (JP)

(73) Assignee: OLYMPUS CORPORATION, Tokyo (JP)

(21) Appl. No.: 15/065,966

(22) Filed: Mar. 10, 2016

(51) Int. Cl. A61B 17/32; A61B 18/04

(52) U.S. Cl. CPC A61B 17/320092; A61B 18/04; A61B 2017/00951

(57) ABSTRACT

A surgical treatment instrument includes an insertion portion including a longitudinal axis, an operation portion provided on one end side of the insertion portion, and a treatment portion provided on the other end side of the insertion portion and configured to treat a living tissue. The treatment portion includes a piezoelectric element configured to generate ultrasound and an ultrasound transmitting material configured to transmit the ultrasound generated by the piezoelectric element to the living tissue.

### STATEMENT REGARDING PRIOR DISCLOSURES

Japanese Patent Application Laid-Open Publication No. 2015-043879 was laid open in Japan on Mar. 12, 2015 from Japanese Patent Application No. 2013-176969, filed on Aug. 28, 2013. The disclosure in Japanese Patent Application Laid-Open Publication No. 2015-043879 was made by Hiroshi ITO, who is the sole inventor of Japanese Patent Application No. 2013-176969.

Hiroshi ITO and Hikaru JINBO are joint inventors in the Subject application.

The disclosure in Japanese Patent Application Laid-Open Publication No. 2015-043879 is a grace period disclosure relative to the subject application.

### BACKGROUND OF THE INVENTION

1. Field of the Invention

The present invention relates to a surgical treatment instrument and a surgical treatment instrument apparatus used in an endoscopic surgical operation and, more particularly, to a surgical treatment instrument and a surgical treatment instrument apparatus that grasp and coagulate/dissect a living tissue.

2. Description of the Related Art

As one of treatment instruments used in an endoscopic surgical operation, there has been known, for example, an ultrasound coagulating/dissecting apparatus functioning as a surgical treatment apparatus that coagulates/dissects a living tissue with ultrasound vibration disclosed in Japanese Patent Application Laid-Open Publication No. 2000-287989. Further, for example, Japanese Patent Application Laid-Open Publication No. 2003-135479 discloses a surgical treatment apparatus in which a heat generating element, which generates heat with electric resistance, is provided at a distal end of a probe, which transmits ultrasound vibration. The surgical treatment apparatus coagulates/dissects a living tissue with the heat from the heat generating element and the ultrasound vibration.

In the ultrasound treatment instruments in the past disclosed in Japanese Patent Application Laid-Open Publication No. 2000-287989 and Japanese Patent Application Laid-Open Publication No. 2003-135479, the ultrasound vibration is transmitted from a bolt-clamped Langevin transducer functioning as an ultrasound vibration source provided in a handle unit to a rigid probe made of a metal material manufactured to be capable of propagating ultrasound.

In the ultrasound treatment instruments in the past, the ultrasound vibration is transmitted to a grasping section provided at the probe distal end. By grasping a living part desired to be treated, the ultrasound treatment instruments can vibrate the probe in the longitudinal direction and perform coagulation/dissection of the grasped living part.

### SUMMARY OF THE INVENTION

A surgical treatment instrument according to an aspect of the present invention includes: an insertion portion extending along a longitudinal axis; an operation portion provided on one end side of the insertion portion along the longitudinal axis; and a treatment portion provided on the other end side of the insertion portion along the longitudinal axis. The treatment portion includes: a piezoelectric element configured to generate ultrasound to treat a living tissue; and an ultrasound transmitting material configured to transmit the ultrasound generated by the piezoelectric element to the living tissue.

A surgical treatment instrument apparatus according to another aspect of the present invention includes: the surgical treatment instrument; a driving-signal generating circuit configured to output a driving signal to the piezoelectric element; and a controller configured to select one of a first mode for controlling a frequency of the driving signal based on a first resonance frequency of the piezoelectric element and a second mode for controlling the frequency of the driving signal based on a second resonance frequency of the treatment portion instrument.

The above and other objects, features and advantages of the invention will become more clearly understood from the following description referring to the accompanying drawings.

### BRIEF DESCRIPTION OF THE DRAWINGS

FIG. 1 is a side view showing a configuration of ultrasound coagulation/dissection forceps functioning as a surgical treatment instrument according to a first embodiment of the present invention;

FIG. 2 is a side view showing a configuration of a treatment portion in the first embodiment;

FIG. 3 is a sectional view showing a configuration of an ultrasound probe cut out along a III-III line in FIG. 2 in the first embodiment;

FIG. 4 is a perspective view showing a partial cross section of the ultrasound probe provided with the piezoelectric element unit in the first embodiment;

FIG. 5 is an exploded perspective view showing the configuration of the piezoelectric element unit in the first embodiment;

FIG. 6 is a perspective view showing a configuration of a piezoelectric element unit in the first embodiment;

FIG. 7 is a block diagram for explaining a controller example of an external apparatus that drives the ultrasound coagulation/dissection forceps in the first embodiment;

FIG. 8 is a block diagram for explaining a controller example of an external apparatus that drives an ultrasound coagulation/dissection forceps an aspect different from an aspect shown in FIG. 7 in the first embodiment;

FIG. 9 is a perspective view showing a configuration of a piezoelectric element unit that longitudinally vibrates in a first modification of the first embodiment;

FIG. 10 is a perspective view showing a configuration of a piezoelectric element unit that laterally vibrates along a longitudinal direction in the first modification of the first embodiment;

FIG. 11 is a perspective view showing a configuration of a piezoelectric element unit that laterally vibrates along a latitudinal direction in the first modification of the first embodiment;

FIG. 12 is an exploded perspective view showing a configuration of a piezoelectric element unit in which a rectangular piezoelectric body is divided in a second modification of the first embodiment;

FIG. 13 is a perspective view showing a configuration of the piezoelectric element unit in which the rectangular piezoelectric body is divided in the second modification of the first embodiment;

FIG. 14 is a block diagram for explaining a controller example of an external apparatus that drives ultrasound coagulation/dissection forceps in the second modification of the first embodiment;

FIG. 15 is a perspective view showing a configuration of a surgical operation portion device functioning as a surgical treatment instrument according to a second embodiment of the present invention;

FIG. 16 is a perspective view showing a configuration of a probe distal end portion in the second embodiment;

FIG. 17 is a perspective view showing a configuration of a probe in the second embodiment;

FIG. 18 is a diagram for explaining a state in which a living tissue is coagulated/dissected in the second embodiment;

FIG. 19 is a diagram of a process for coagulating/dissecting the living tissue in the second embodiment;

FIG. 20 is a diagram of a state in which the living tissue is coagulated/dissected in the second embodiment;

FIG. 21 is an exploded perspective view showing a configuration of a probe in a modification of the second embodiment;

FIG. 22 is a perspective view showing a configuration of a hand piece functioning as a surgical treatment instrument according to a third embodiment of the present invention; and

FIG. 23 is a partial sectional view showing a configuration of a distal end portion of the hand piece in the third embodiment.

### DETAILED DESCRIPTION OF THE PREFERRED EMBODIMENTS

The present invention is explained below with reference to the drawings.

Note that, in the following explanation, that the drawings based on respective embodiments are schematic and relations between thicknesses and widths of respective portions, ratios of the thicknesses of the respective portions, and the like are different from real ones. Portions, relations and ratios of dimensions of which are different from one another, are sometimes included among the drawings.

### First Embodiment

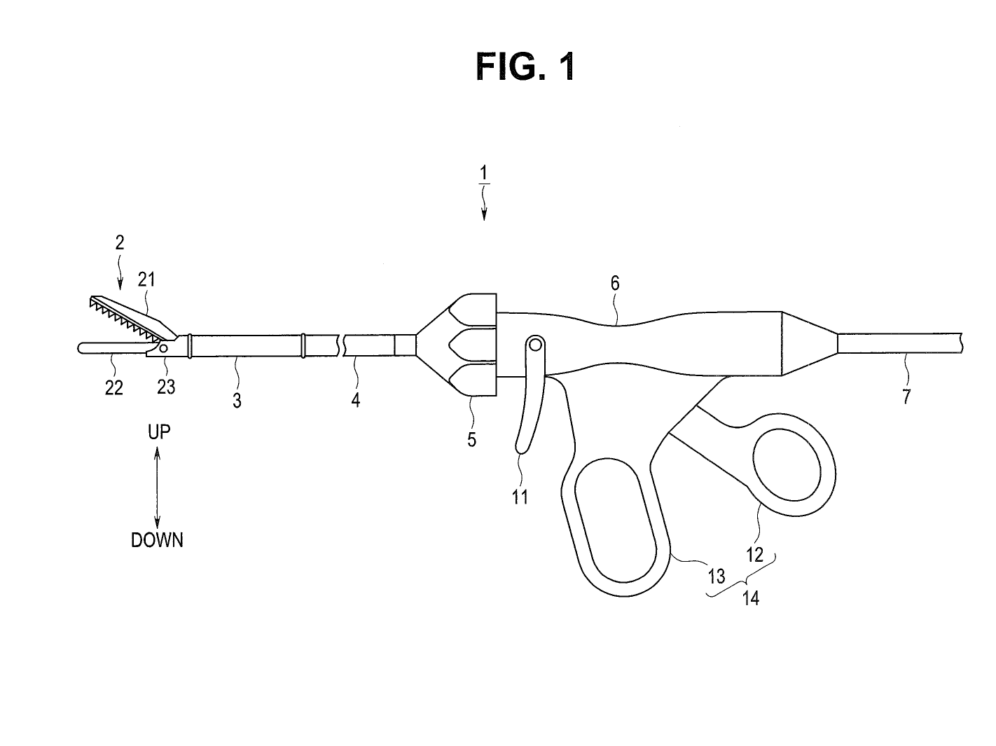

First, a first embodiment of the present invention is explained below with reference to FIGS. 1 to 14.

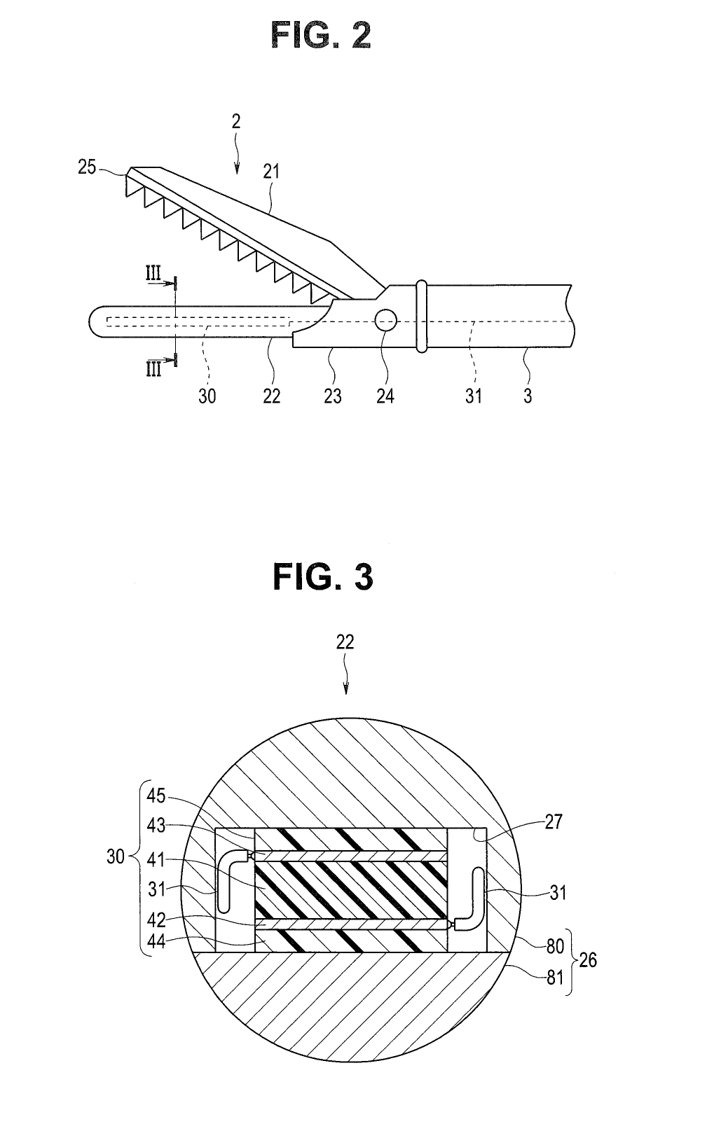

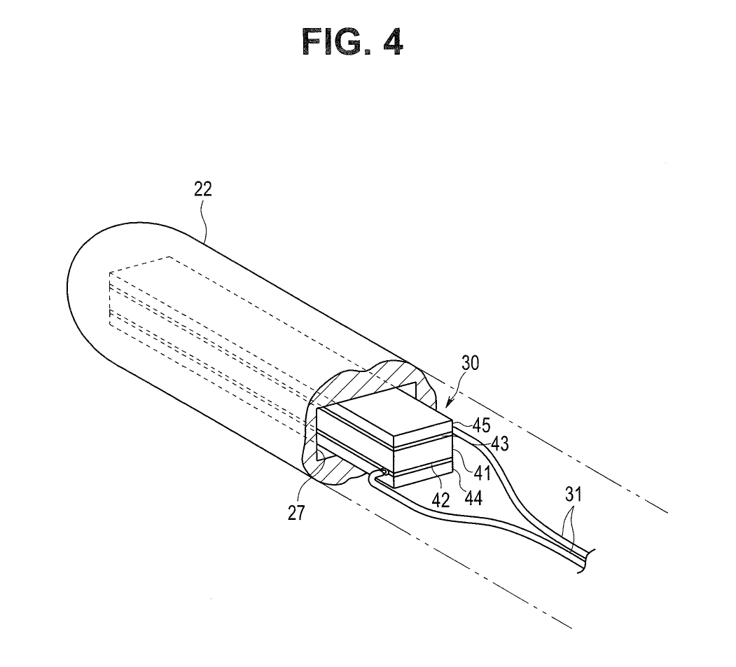

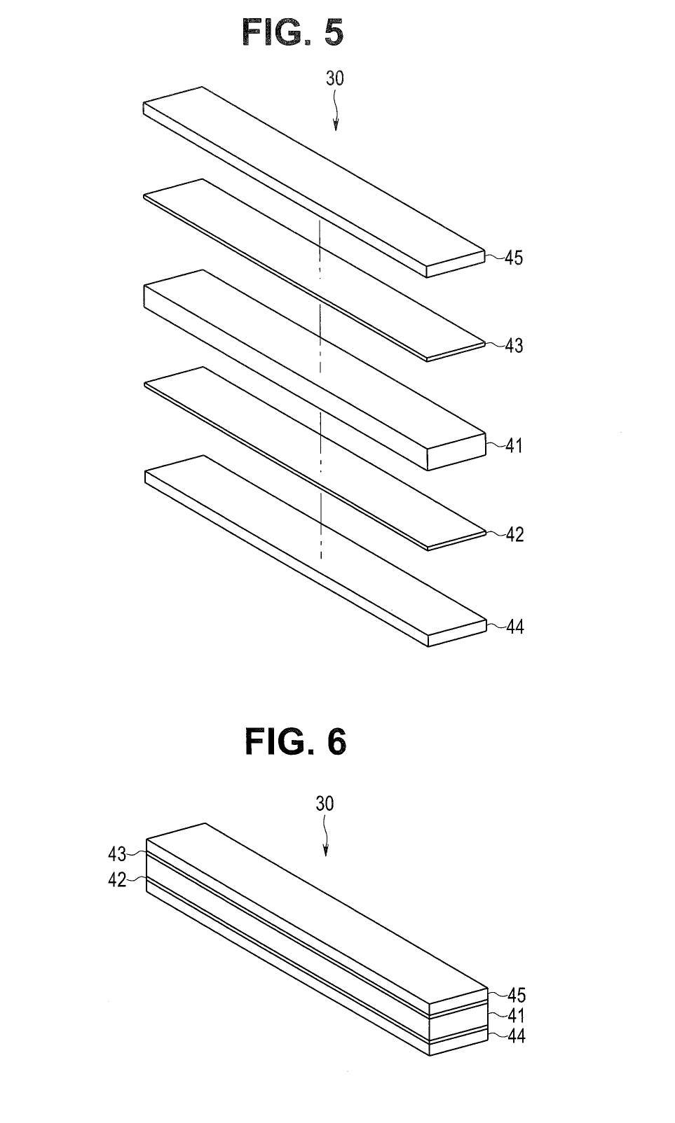

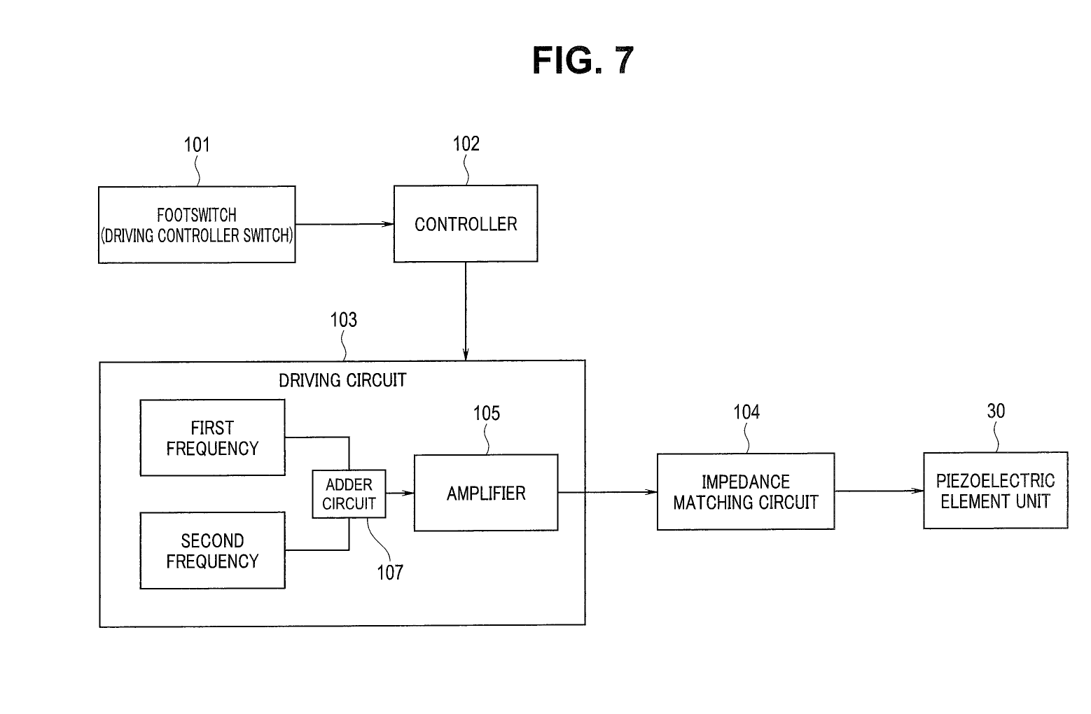

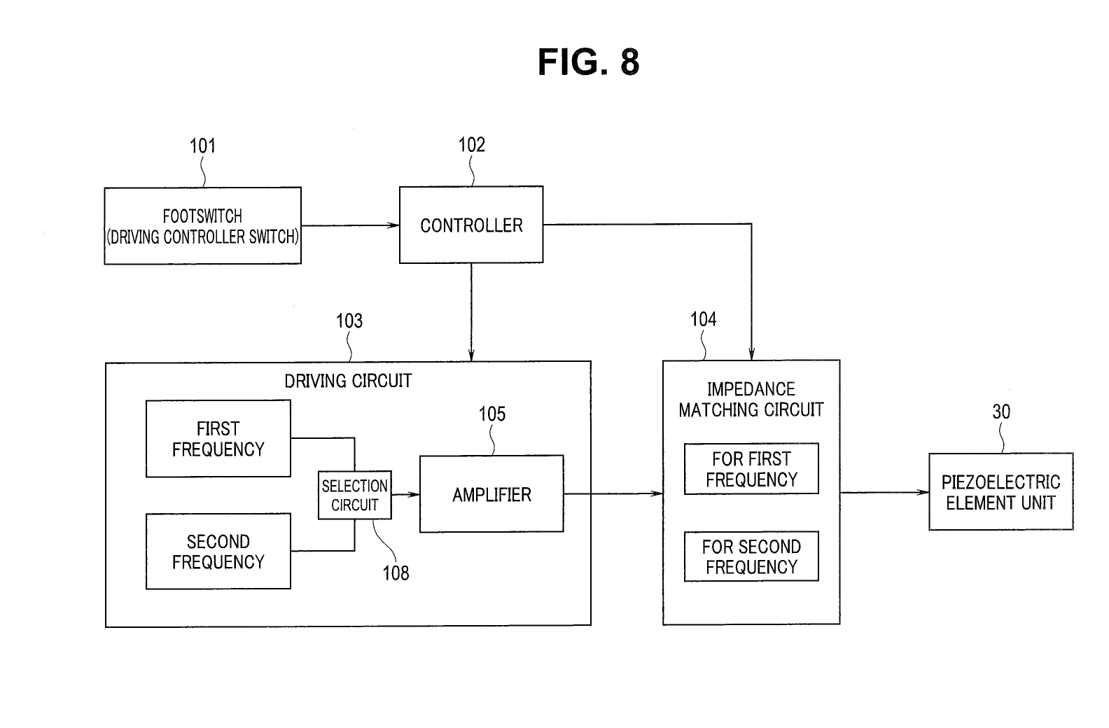

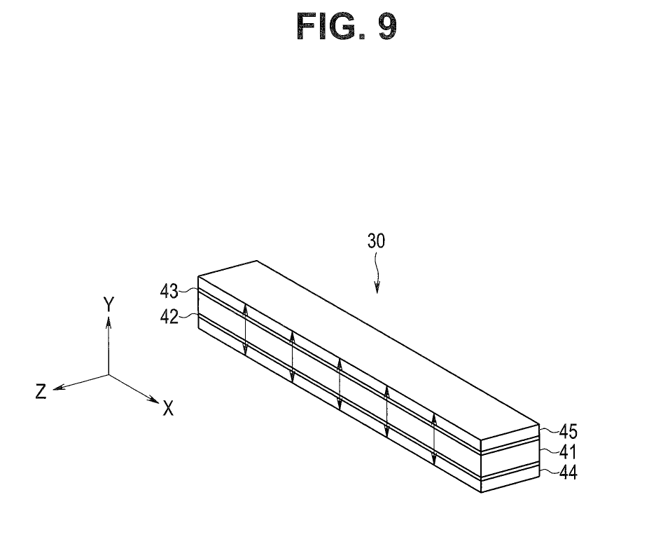

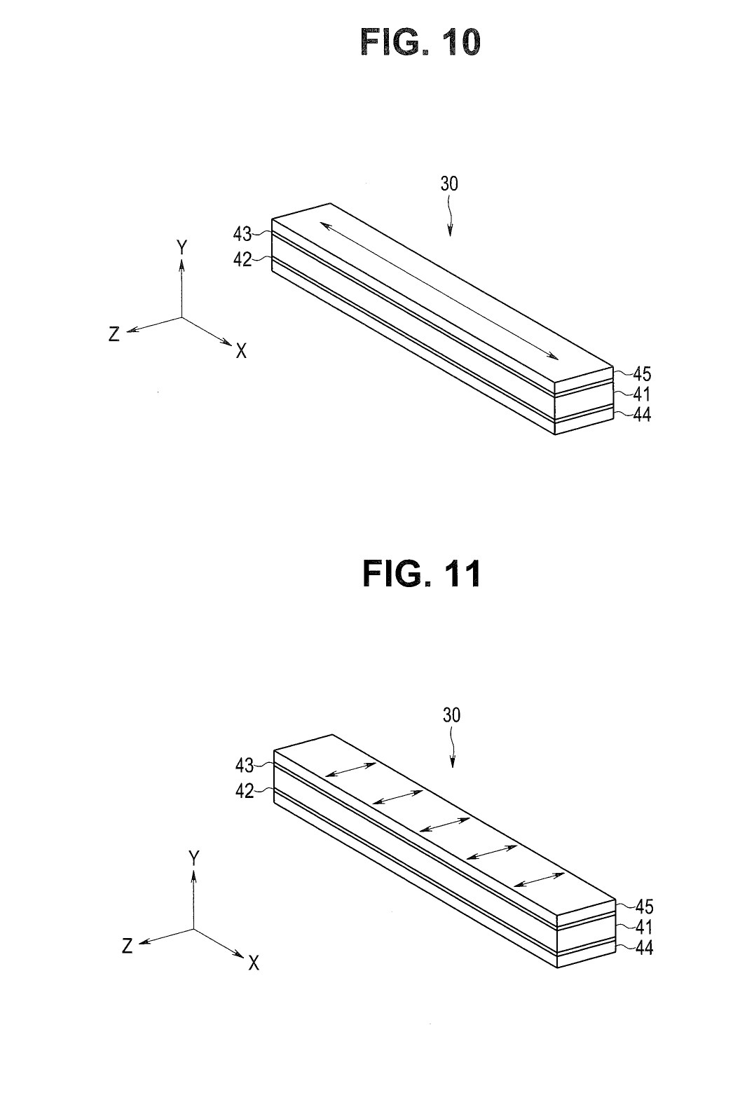

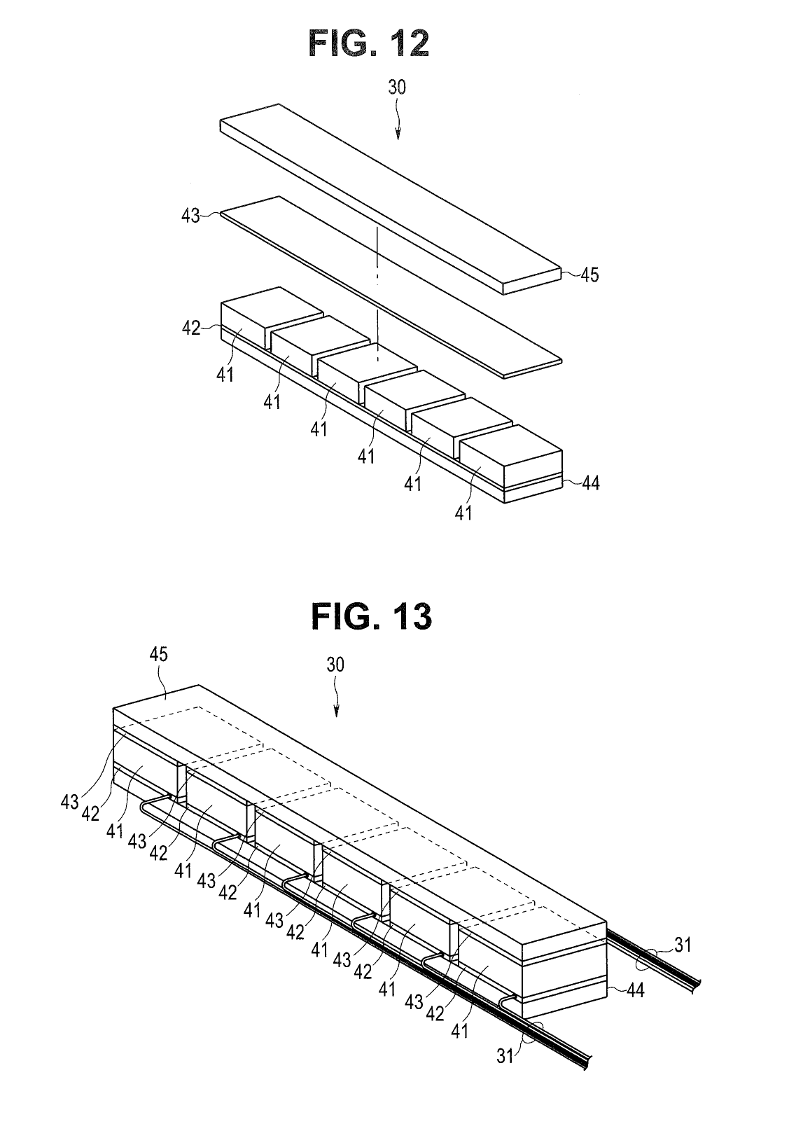

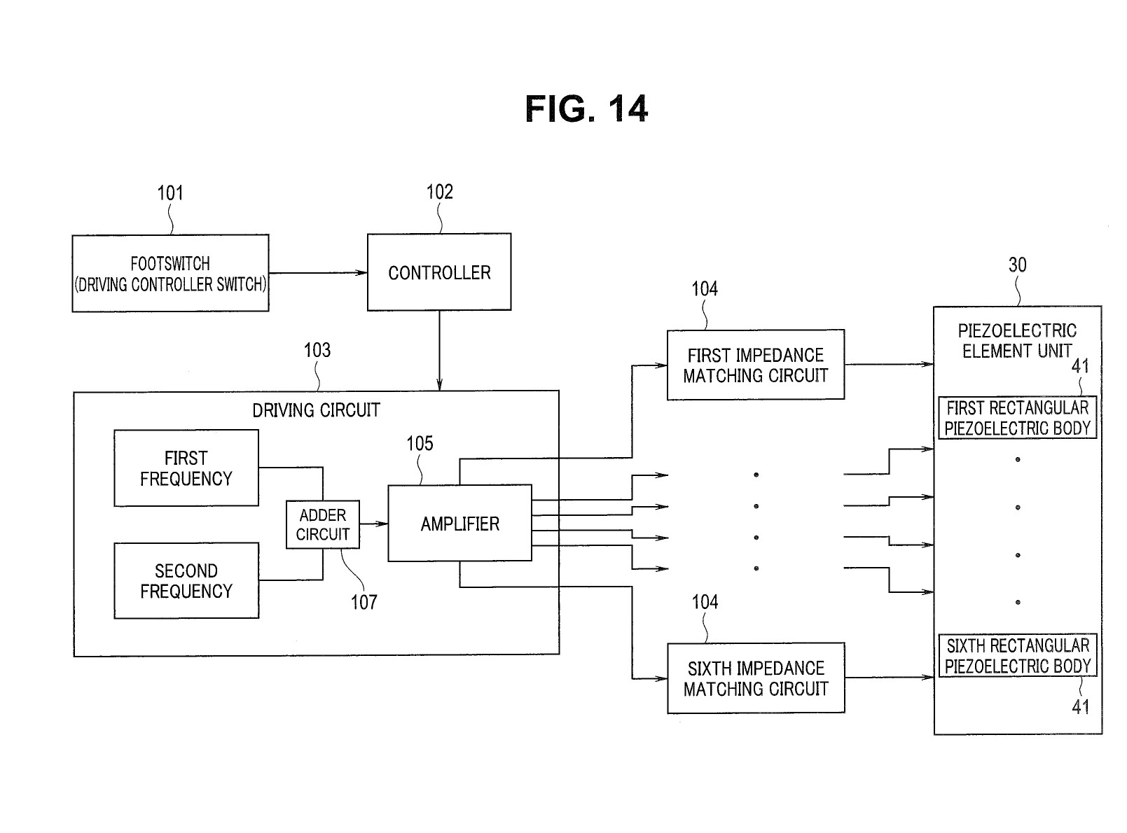

FIG. 1 is a side view showing a configuration of ultrasound coagulation/dissection forceps functioning as a surgical treatment instrument. FIG. 2 is a side view showing a configuration of a treatment portion. FIG. 3 is a sectional view showing a configuration of an ultrasound probe cut out along a line in FIG. 2. FIG. 4 is a perspective view showing a partial cross section of the ultrasound probe provided with the piezoelectric element unit. FIG. 5 is an exploded perspective view showing the configuration of the piezoelectric element unit. FIG. 6 is a perspective view showing a configuration of a piezoelectric element unit. FIG. 7 is a block diagram for explaining a controller example of an external apparatus that drives the ultrasound coagulation/dissection forceps. FIG. 8 is a block diagram for explaining a controller example of an external apparatus that drives the ultrasound coagulation/dissection forceps in an aspect different from an aspect shown in FIG. 7. FIG. 9 is a perspective view showing a configuration of a piezoelectric element unit that longitudinally vibrates in a first modification. FIG. 10 is a perspective view showing a configuration of a piezoelectric element unit that laterally vibrates along a longitudinal direction in the first modification. FIG. 11 is a perspective view showing a configuration of a piezoelectric element unit that laterally vibrates along a latitudinal direction in the first modification. FIG. 12 is an exploded perspective view showing a configuration of a piezoelectric element unit in which a rectangular piezoelectric body is divided in a second modification. FIG. 13 is a perspective view showing a configuration of the piezoelectric element unit in which the rectangular piezoelectric body is divided in the second modification. FIG. 14 is a block diagram for explaining a controller example of an external apparatus that drives ultrasound coagulation/dissection forceps in the second modification.

### (Surgical Treatment Instrument)

First, configurations of a surgical treatment instrument and a surgical treatment instrument apparatus including the surgical treatment instrument according to the present embodiment are explained below.

FIG. 1 is a diagram of ultrasound coagulation/dissection forceps 1 functioning as the surgical treatment instrument according to the present embodiment. The ultrasound coagulation/dissection forceps 1 mainly include, in order from a distal end, a treatment portion 2, a bending portion 3 concatenated to the treatment portion 2, an insertion tube 4 concatenated to the bending portion 3, a rotation operation portion member 5 to which a proximal end portion of the insertion tube 4 is connected, a housing 6 in which the rotation operation portion member 5 is turnably provided, and a cable 7 extending from the housing 6, a connector (not shown in the figure) detachably attachable to an external apparatus (not shown in the figure) being disposed at an extension end of the cable 7. Note that an insertion portion is configured by at least one of the bending portion 3 and the insertion tube 4. Then, the bending portion 3 and the insertion tube 4 can be configured to be insertion pipe.

In the housing 6, a bending portion operation portion lever 11 for bending the bending portion 3 and a handle 14 including a movably operable operation portion handle 12 and a fixed handle 13 are disposed.

The bending portion 3 is configured to bend, according to turning operation portion of the bending portion operation portion lever 11, in upward and downward two directions (UP and DOWN directions in the figure) viewed toward a paper surface when the bending portion 3 is present in a neutral position explained below. Note that the bending portion 3 may be configured to bend in four directions including not only the upward and downward directions but also right and left directions.

The treatment portion 2 connected to a distal end of the bending portion 3 includes a jaw 21 that moves up and down by operating the operation portion handle 12, an ultrasound probe 22 functioning as an ultrasound blade that grasps and coagulates/dissects a living tissue according to opening and closing of the jaw 21, and a holding member 23 that turnably holds the jaw 21 and fixes a proximal end portion of the ultrasound probe 22.

Note that a not-shown flexible coil pipe of the bending portion 3 is coupled to a proximal end portion of the jaw 21 housed in the holding member 23. The coil pipe is inserted through and disposed in the bending portion 3 and the insertion tube 4 and coupled to the operation portion handle 12 rotatably attached to the housing 6.

When the operation portion handle 12 is operated back and forth, the coil pipe advances and retracts in association with the operation portion. The jaw 21 operates around a support shaft provided in the holding member 23. The jaw 21 turns in upward and downward directions in which the jaw 21 comes into contact with and separates from the ultrasound probe 22. Consequently, it is possible to grasp the living tissue between the jaw 21 and the ultrasound probe 22.

Note that the coil pipe has flexibility for preventing a bending portion motion of the bending portion 3 from being hindered. The coil pipe is configured not to generally extend and contract in a longitudinal axial direction when the coil pipe is advanced and retracted to open and close the jaw 21.

In the bending portion 3, the insertion tube 4, and the rotation operation portion member 5, inner holes communicating with an inside of the housing 6 are formed. A bending portion operation portion wire for pulling and loosening a plurality of bending portion pieces (not shown in the figure) provided in the bending portion 3, the coil pipe for opening and closing the jaw 21, and the like are inserted through the inner holes.

The insertion tube 4 is configured by a rigid metal pipe. The insertion tube 4 can be turned to the right and the left in a range of approximately half rotation (180°) in the longitudinal axial direction by turning, from the neutral position, the rotation operation portion member 5 connected and fixed to a proximal end portion. Consequently, the treatment portion 2 and the bending portion 3 are also rotated around a longitudinal axis of the insertion tube 4 in association with the turning of the insertion tube 4.

That is, the ultrasound coagulation/dissection forceps 1 are configured to be capable of, according to the turning operation portion of the rotation operation portion member 5, tilting the treatment portion 2 in a direction in which the living tissue is easily grasped and tilting the two directions in which the bending portion 3 bends.

### (Treatment Portion)

A configuration of the treatment portion 2 in the present embodiment is explained below.

As shown in FIG. 2, the jaw 21 of the treatment portion 2 is assembled by coaxially pivotally attaching proximal end to the holding member 23 with a pin 24.

A proximal end portion of the ultrasound probe 22 of the treatment portion 2 is fixed in the holding member 23. The ultrasound probe 22 is shaped in a substantially columnar shape to project forward. The ultrasound probe 22 is disposed to be opposed to the jaw 21 that opens and closes.

A piezoelectric element unit 30 explained below is incorporated in the ultrasound probe 22. The piezoelectric element unit 30 is inserted through and secured to the cable 7 from the housing 6 via the inner holes of the bending portion 3, the insertion tube 4, and the rotation operation portion member 5 and connected to an electric cable 31 that supplies driving power.

Note that, the jaw 21 includes a pressing member 25 that made of an insulative member, is provided in a position opposed to the ultrasound probe 22 during the opening and closing. The pressing member 25 projects from the jaw 21. A distal end of the pressing member 25 is shaped in a recessed and protruded teeth shape.

In this way, since the pressing member 25 is provided in the jaw 21, the treatment portion 2 is configured to prevent the living tissue grasped by the treatment portion 2 and the ultrasound probe 22 from easily slipping off. That is, in the treatment portion 2 and the jaw 21 of the treatment portion 2, since the pressing member 25 is provided in the jaw 21, a grasping for grasping the living tissue is configured by the ultrasound probe 22.

Note that, the ultrasound probe 22 may further comprise a heat generation pattern functioning as a heat generating portion element for assisting coagulation treatment portion of the living tissue.

The ultrasound probe 22 functions as an ultrasound transmitting material that transmits ultrasound generated from the piezoelectric element unit (a piezoelectric element) 30 explained below to at least the pressing member 25.

### (Piezoelectric Element Unit)

The piezoelectric element unit (the piezoelectric element) 30 functioning as an ultrasound transducer is explained below.

As shown in FIGS. 3 and 4, the piezoelectric element unit 30 provided on an inside of the ultrasound probe 22 is disposed such that upper and lower parts of the piezoelectric element unit 30 are in surface contact with a hole 27 having a rectangular shape in formed in grasping portion metal 26 functioning as an ultrasound transmitting material having a substantially columnar shape made of duralumin or a titanium alloy such as 64Ti that forms an exterior of the ultrasound probe 22. That is, in the ultrasound probe 22, the grasping portion metal 26 configures a front mass 80 and a back mass 81 for the piezoelectric element unit 30. The front mass 80 and the back mass 81 have a function of transmitting ultrasound generated by the piezoelectric element unit 30.

In this way, in the ultrasound coagulation/dissection forceps 1 according to the present embodiment, the piezoelectric element unit 30 is provided on an inside of the grasping portion metal 26 of the ultrasound probe 22, which is one of the components configuring the grasping portion of the treatment portion 2.

As shown in FIGS. 5 and 6, the piezoelectric element unit 30 includes a rectangular piezoelectric body 41 having a rectangular plate shape on a surface thereof, a positive-side electrode plate 42 functioning as a positive electrode layer and a negative-side electrode plate 43 functioning as a negative electrode layer, which are metal plates of copper or the like stacked to sandwich the rectangular piezoelectric body 41, and two insulating plates 44 and 45 stacked to further sandwich the electrode plates 42 and 43. Note that a material for the insulating plates 44 and 45 having high thermal conductivity and fewer mechanical losses is suitable. Alumina or the like is used as the insulating plates 44 and 45.

In the piezoelectric element unit 30, the rectangular piezoelectric body 41, the respective electrode plates 42 and 43, and the respective insulating plates 44 and 45 are stacked such that four corners and four sides thereof coincide with one another. The piezoelectric element unit 30 is formed in a substantially square pole shape as a whole. That is, shapes of front and rear surfaces of the rectangular piezoelectric body 41, the respective electrode plates 42 and 43, and the respective insulating plates 44 and 45 are shaped in substantially the same rectangular shapes.

Note that the rectangular piezoelectric body 41, the respective electrode plates 42 and 43, and the respective insulating plates 44 and 45 are bonded and integrated by brazing. Base metal showing satisfactory adhesion to a brazing material and a base material is formed on the surfaces of the rectangular piezoelectric body 41, the respective electrode plates 42 and 43, and the respective insulating plates 44 and 45 according to necessity. As the brazing material, gold tin (AuSn) or solder is used as a material having heat resistance and high thermal conductivity that does not melt even during coagulation/dissection treatment portion. A wire of the electric cable 31 is electrically connected to the positive-side electrode plate 42 and the negative-side electrode plate 43 also using gold tin (AuSn), solder, or the like. A brazing material having a certain thickness may be used as the electrode plates 42 and 43.

Note that the bonding of the rectangular piezoelectric body 41, the respective electrode plates 42 and 43, and the respective insulating plates 44 and 45 configures the piezoelectric element unit 30 functioning as a device used in the ultrasound coagulation/dissection forceps 1 functioning as a surgical treatment instrument that performs treatment portion using ultrasound. Therefore, the bonding needs to have heat resistance and durability.

Therefore, for a metal-based bonding material used as the brazing material, besides gold tin (AuSn), metal nano-particle bonding or the like may be used. Note that, when solder is used, it is desirable to use solder for high temperature having a melting point equal to or higher than at least 200° C. and desirably equal to or higher than 300° such as aluminum-based solder or gold-based solder.

Further, the bonding material is not limited to the metal material. For example, a ceramics adhesive may be used.

In this way, in the piezoelectric element unit 30, the insulating plate 44, the positive-side electrode plate 42, the rectangular piezoelectric body 41, the negative-side electrode plate 43, and the insulating plate 45 are stacked in this order such that the grasping portion metal 26 is insulated from a driving current flowing to the positive-side electrode plate 42 and returning to the negative-side electrode plate 43. The grasping portion metal 26 and the piezoelectric element unit 30 are configured to be capable of transmitting ultrasound vibration and capable of being thermally combined.

The grasping portion metal 26 is configured from two members, that is, grasping-portion-metal forming members 80 and 81 configuring the front mass or the back mass. When the piezoelectric element unit 30 is secured to the grasping-metal forming members 80 and 81 by brazing, the grasping-portion-metal forming members 80 and 81 are also brazed to form the hole 27. Note that the grasping portion metal 26 needs to have biocompatibility and desirably has high thermal conductivity.

That is, the piezoelectric element unit 30 is surrounded by the grasping-portion-metal forming members 80 and 81 configuring the front mass and the back mass and is disposed to be separated from the grasping-portion-metal forming members 80 and 81 in a width direction.

In the grasping portion metal 26, in order to increase strength and prevent a break, a filler is desirably filled in a gap of separation from the piezoelectric element unit 30 in a width direction of the hole 27 formed by the grasping-portion-metal forming members 80 and 81 in a state in which the piezoelectric element unit 30 is fixed.

As the filler, an epoxy-based high heat resistant resin or a ceramics-based inorganic filler having heat resistance of 300° C. is used.

Incidentally, a piezoelectric characteristic of the piezoelectric element unit 30 needs to be not deteriorated even at temperature for performing coagulation/dissection and even when the respective members of the rectangular piezoelectric body 41, the respective electrode plates 42 and 43, and the respective insulating plates 44 and 45 are brazed. Further, the treatment portion 2 is considered to have temperature equal to or higher than 200° C. during coagulation/dissection treatment portion of the living tissue. Temperature equal to or higher than 200° C. is applied to the treatment portion 2 during solder bonding and temperature equal to or higher than 300° C. is applied to the treatment portion 2 during gold tin (AuSn) bonding.

Therefore, when lead zirconate titanate (PZT) generally in use is used in the rectangular piezoelectric body 41 functioning as a piezoelectric material, a Curie point of approximately 300° C. is not sufficiently high temperature. The piezoelectric characteristic is deteriorated during the bonding process and as time of use elapses. As a piezoelectric material having durability against these temperatures, there is, for example, a lithium niobate single crystal (LiNbO3), which is a piezoelectric single crystal, having a Curie point of approximately 1200° C.

In crystal orientation called 36-degree rotated Y cut of the lithium niobate single crystal (LiNbO3), an electromechanical coupling coefficient kt in a thickness direction of a transducer has a value of the same degree as that of lead zirconate titanate (PZT). An electric signal can be efficiently converted into ultrasound vibration.

A value of the electromechanical coupling coefficient kt changes according to crystal orientation and maximizes at a certain angle. Specific crystal orientation, for example, 36-degree rotated Y cut is explained as an example here taking into account easiness of acquisition in a market. However, the crystal orientation is not limited to this and may be crystal orientation near the specific crystal orientation in a range in which the electromechanical coupling coefficient does not greatly decrease. The same applies to vibration in a lateral direction explained below.

Further, the lithium niobate single crystal (LiNbO3) is one of non-lead piezoelectric materials having high mechanical Q values suitable for an ultrasound transducer for high power use. Since lead is not used, the lithium niobate single crystal (LiNbO3) is excellent in environmental properties.

### (Driving Controller)

Driving controller by an external apparatus that drives the ultrasound coagulation/dissection forceps 1 according to the present embodiment configured as explained above is explained below. Note that a surgical treatment instrument system, functioning as an ultrasound coagulating/dissecting apparatus, is configured by the ultrasound coagulation/dissection forceps 1 and the external apparatus.

As shown in FIG. 7, in the external apparatus not shown in the figure to which the ultrasound coagulation/dissection forceps 1 is connected, a footswitch 101 functioning as a driving controller switch for operating the ultrasound probe 22 is provided and a controller 102, a driving circuit 103, an impedance matching circuit 104, and the like are provided as controller systems. Note that the driving controller switch is not limited to the footswitch 101. An operation portion button may be provided in the housing 6 of the ultrasound coagulation/dissection forceps 1.

A controller example by the external apparatus is briefly explained. When a signal is supplied to the controller 102 according to operation portion of the footswitch 101, the controller 102 outputs a controller signal to the driving circuit 103. Note that the driving circuit 103 includes an amplifier 105 that receives an input of sine wave signals having a first frequency and a second frequency, which are resonant frequencies, and amplifies the sine wave signals and an adder circuit 107 that mixes the sine wave signals having the two frequencies (the first frequency and the second frequency) at amplitude and any ratio set in advance.

When the footswitch 101 is operated, a controller signal is outputted to the driving circuit 103 via the controller 102, which supplies a driving signal serving as an electric signal to the piezoelectric element unit 30, by the controller 102, which is one of controller systems.

The driving circuit 103, which is one of the controller systems, mixes the sine wave signals having the two frequencies, which are a sine wave having the resonance frequency (the first frequency) determined by a transducer alone in the rectangular piezoelectric body 41 and a sine wave having the resonance frequency (the second frequency) determined by the ultrasound probe 22 functioning as a structure including the grasping portion metal 26, the respective electrode plates 42 and 43, and the insulating plates 44 and 45 besides the rectangular piezoelectric body 41, at the amplitude and any ratio set in advance by the adder circuit 107, then amplifies the sine wave signals with the amplifier 105, and outputs the sine wave signals to the impedance matching circuit 104. The sine wave signals are outputted from the impedance matching circuit 104, which covers two frequency domains, to the positive-side electrode plate 42 of the piezoelectric element unit 30.

Note that, as shown in FIG. 8, a configuration may be adopted in which a selection circuit 108 is provided that selects one of the sine wave having the first frequency and the sine wave having the second frequency in the driving circuit 103 according to a signal from the controller 102, one of the sine waves is selected by the selection circuit 108, and a circuit block for the first frequency or a circuit block for the second frequency suitable for a frequency selected in the impedance matching circuit 104 as well is selected.

In this way, the piezoelectric element unit 30 is driven when a driving signal obtained by mixing the sine wave signals having the two frequencies (the first frequency and the second frequency) at any ratio is supplied from the driving circuit 103 to the piezoelectric element unit 30 via the impedance matching circuit 104.

Consequently, the entire ultrasound probe 22, which is a structure incorporating the piezoelectric element unit 30, performs ultrasound vibration. At this point, the rectangular piezoelectric body 41 of the piezoelectric element unit 30 generates heat. The heat is transmitted from the respective electrode plates 42 and 43 and the insulating plates 44 and 45 to the grasping portion metal 26 and acts on the living tissue grasped by the treatment portion 2. Coagulation/dissection is performed.

Incidentally, in the ultrasound coagulation/dissection forceps 1, when the resonance frequency (the first frequency) of the rectangular piezoelectric body 41 alone and the resonance frequency (the second frequency) of the entire ultrasound probe 22, which is a stacked structure, are compared, the resonance frequency (the second frequency) of the entire ultrasound probe 22 is lower than the resonance frequency (the first frequency) of the rectangular piezoelectric body 41 alone. Therefore, when the piezoelectric element unit 30 is driven with the same electric power, vibration amplitude is relatively larger than when the piezoelectric element unit 30 is driven at the resonance frequency (the second frequency) of the entire ultrasound probe 22. A ratio of dissection by mechanical action of ultrasound vibration is relatively predominant.

Conversely, in the ultrasound coagulation/dissection forceps 1, when the piezoelectric element unit 30 is driven at the resonance frequency (the first frequency) of the rectangular piezoelectric body 41 alone, vibration amplitude is relatively smaller than when the piezoelectric element unit 30 is driven at the resonance frequency (the second frequency) of the entire ultrasound probe 22. A ratio of coagulation by heat is relatively predominant.

According to such characteristics, by configuring the external apparatus, which supplies the driving signal to the ultrasound coagulation/dissection forceps 1, to be capable of adjusting an amplitude mixing ratio of two frequency components (the first frequency and the second frequency) of the driving signal supplied to the rectangular piezoelectric body 41, it is possible to select, according to operation portion by a surgeon, a state in which hemostatic performance for the living tissue by the treatment portion 2 of the ultrasound coagulation/dissection forceps 1 is high or a state in which dissection performance for the living tissue by the treatment portion 2 of the ultrasound coagulation/dissection forceps 1 is high. Note that the selection of the state in which hemostatic performance for the living tissue by the treatment portion 2 of the ultrasound coagulation/dissection forceps 1 is high or the state in which dissection performance for the living tissue by the treatment portion 2 of the ultrasound coagulation/dissection forceps 1 is high can be operated by the footswitch 101, which is the driving controller switch.

As explained above, in the ultrasound coagulation/dissection forceps 1 according to the present embodiment, the piezoelectric element unit 30 including the rectangular piezoelectric body 41 made of the lithium niobate single crystal (LiNbO3) having the high Curie point of approximately 1200° C. is incorporated in the ultrasound probe 22 configuring the treatment portion 2, which is the grasping portion. The ultrasound coagulation/dissection forceps 1 is configured to perform coagulation/dissection of the living tissue with ultrasound generated by the rectangular piezoelectric body 41 and heat generation of the rectangular piezoelectric body 41 itself.

In the ultrasound coagulation/dissection forceps 1, the rectangular piezoelectric body 41 is provided in the grasping portion metal 26 configuring the exterior of the ultrasound probe 22, which is a portion of the treatment portion 2 functioning as the grasping portion in contact with the living tissue. Therefore, the rectangular piezoelectric body 41 is present right under the living tissue via the grasping portion metal 26. A distance between the rectangular piezoelectric body 41 and the living tissue can be short. Thermal resistance by the grasping portion metal 26 can be small. Generated heat by the rectangular piezoelectric body 41 is easily transmitted to the grasped living tissue.

By adopting such a configuration, the ultrasound coagulation/dissection forceps 1 does not need to be configured as a rigid long ultrasound probe of a configuration in the past by providing the piezoelectric element unit 30 in the housing 6 such that ultrasound vibration can be transmitted from the piezoelectric element unit 30 to the insertion tube 4, which is a long. The bending portion 3 can be provided between the treatment portion 2 and the insertion tube 4 such that the treatment portion 2 on a distal end side can be bent or swung. A direction of the treatment portion 2 can be changed by the bending portion 3.

In the ultrasound coagulation/dissection forceps 1, there is a possibility to decrease a performance of coagulation/dissection with just disposing the rectangle piezoelectric element 41 into the piezoelectric element unit 30 as grasping portion of treatment portion 2. Because the rectangular piezoelectric body 41 is smaller and the displacement of vibration is smaller than a conventional configuration.

However, the ultrasound coagulation/dissection forceps 1 according to the present embodiment uses not only the ultrasound vibration generated by the rectangular piezoelectric body 41 but also the heat generated when the rectangular piezoelectric body 41 is driven. That prevents from deteriorating the coagulation/dissection performance for the living tissue even with smaller amplitude of the ultrasound vibration, compared to the conventional ultrasound treatment portion instrument.

That is, in the ultrasound coagulation/dissection forceps 1, when high electric power is inputted to the rectangular piezoelectric body 41 to cause the rectangular piezoelectric body 41 to perform ultrasound vibration, not only large vibration but also heat in the rectangular piezoelectric body 41 itself is generated. By adopting a configuration in which thermal resistance from the rectangular piezoelectric body 41 to a portion in contact with the living tissue grasped by the treatment portion 2 is reduced, the heat generated by the rectangular piezoelectric body 41 is transmitted to the grasped living tissue and the coagulation/dissection performance is improved.

As explained above, a surgical treatment portion system including the ultrasound coagulation/dissection forceps 1 functioning as an apparatus for surgical treatment portion according to the present invention and the external apparatus can adopt a configuration with high operability that can facilitate approach to a treatment portion part or a diseased part to make it easy to grasp the living tissue and can quickly and easily apply appropriate coagulation treatment portion or dissection treatment portion to the living tissue.

Further, as a method of generating portion ultrasound vibration with the piezoelectric element unit 30, there are two methods, that is, a method of using a resonance mode by the first frequency previously mentioned and a method of using a resonance mode by the second frequency, which is a resonance frequency of a structure in which the ultrasound probe 22, which is a blade, and the piezoelectric element unit 30 are combined.

In resonance of the piezoelectric element unit 30, a heat generation amount of the piezoelectric element unit 30 is increased by generation of ultrasound vibration having a frequency in a MHz band due to thickness longitudinal vibration, shear vibration, expansion vibration, or the like. A coagulation property/a hemostatic property for the living tissue is improved.

On the other hand, in the resonance mode by the structure in which the ultrasound probe 22 and the piezoelectric element unit 30 are combined, a vibration amplitude of the ultrasound probe 22 is increased by generation of ultrasound vibration having a frequency in a kHz band due to expansion vibration, shear vibration, torsional vibration, traveling wave vibration, or the like. A dissection property for the living tissue is improved.

Therefore, the ultrasound coagulation/dissection forceps 1 may be configured to be capable of switching two modes, that is the first mode in which the coagulation property/the hemostatic property for the living tissue is improved by the controller 102 of the external apparatus and the second mode in which the dissection property for the living tissue is improved by the controller 102 of the external apparatus.

### (First Modification)

Incidentally, in the above explanation, a vibrating direction of the rectangular piezoelectric body 41 of the piezoelectric element unit 30 is not specified. For example, when a 36-degree rotated Y cut lithium niobate single crystal (LiNbO3) substrate is cut to create the rectangular piezoelectric body 41, longitudinal vibration indicated by an arrow along a thickness direction (a Y direction in the figure) of the rectangular piezoelectric body 41 can be efficiently excited as shown in FIG. 9. When a 163-degree rotated Y cut lithium niobate single crystal (LiNbO3) substrate or an X cut lithium niobate single crystal (LiNbO3) substrate is cut to create the rectangular piezoelectric body 41, lateral vibration indicated by an arrow along a longitudinal direction (an X direction in the figure) of the rectangular piezoelectric body 41 can be efficiently excited as shown in FIG. 10 or lateral vibration indicated by an arrow along a latitudinal direction (a Z direction in the figure) of the rectangular piezoelectric body 41 can be efficiently excited as shown in FIG. 11.

In general, speed of a lateral wave is approximately a half of speed of a longitudinal wave and is low compared with the speed of the longitudinal wave. Therefore, a resonance frequency can be set lower in the lateral wave than in the longitudinal wave even in a structure having the same dimensions. It is easier to increase amplitude. Therefore, the rectangular piezoelectric body 41 desirably uses the lateral vibration in a direction orthogonal to the thickness direction as shown in FIG. 10 or 11. It is possible to increase action of mechanical vibration more than when the longitudinal vibration shown in FIG. 9 is used and further improve the coagulation/dissection performance for the living tissue.

That is, the ultrasound coagulation/dissection forceps 1 has a configuration in which the coagulation/dissection performance for the living tissue by the treatment portion 2 is improved by setting the crystal orientation of the lithium niobate single crystal (LiNbO3) configuring the rectangular piezoelectric body 41 of the piezoelectric element unit 30 such that the rectangular piezoelectric body 41 laterally vibrates.

### (Second Modification)

The lithium niobate single crystal (LiNbO3) configuring the rectangular piezoelectric body 41 has small mechanical strength and is likely to be cracked. Therefore, as shown in FIG. 12, the rectangular piezoelectric body 41 is divided into plural pieces, for example, six pieces. The divided rectangular piezoelectric bodies 41 are provided in parallel to one another. A size of the individual rectangular piezoelectric bodies 41 is reduced. Consequently, it is possible to reduce stress applied to the rectangular piezoelectric bodies 41 during manufacturing, during operation portion, and the like and prevent the rectangular piezoelectric bodies 41 from being easily cracked.

Further, as shown in FIG. 13, the positive-side electrode plate 42 and the negative-side electrode plate 43 may be also divided into a plurality of pieces, for example, six pieces to individually supply driving electric power to each of the rectangular piezoelectric bodies 41. The electric cable 31 is individually connected to each of the divided positive-side electrode plates 42 and the divided negative-side electrode plates 43. Note that the negative-side electrode plate 43 feeds back the driving electric power. Therefore, it is not always necessary to divide the negative-side electrode plate 43. Only the positive-side electrode plate 42 may be divided according to the number of the rectangular piezoelectric bodies 41, that is, six.

In addition, as shown in FIG. 14, the external apparatus, which drives the ultrasound coagulation/dissection forceps 1, may have a configuration including first to sixth impedance matching circuits 104 that supply driving electric power to respective divided first to sixth rectangular piezoelectric bodies 41.

That is, the external apparatus can variously adjust a ratio of the two frequency components (the first frequency and the second frequency) of a supplied driving signal for each of the first to sixth rectangular piezoelectric bodies 41 of the piezoelectric element unit 30. Therefore, it is possible to selectively set various states in which the hemostatic performance for the living tissue by the treatment portion 2 of the ultrasound coagulation/dissection forceps 1 is high or various states in which the dissection performance by the treatment portion 2 of the ultrasound coagulation/dissection forceps 1 is high.

Note that the adder circuit 107 shown in FIG. 14 may be replaced with the selection circuit 108 as another form shown in FIG. 8.

### Second Embodiment

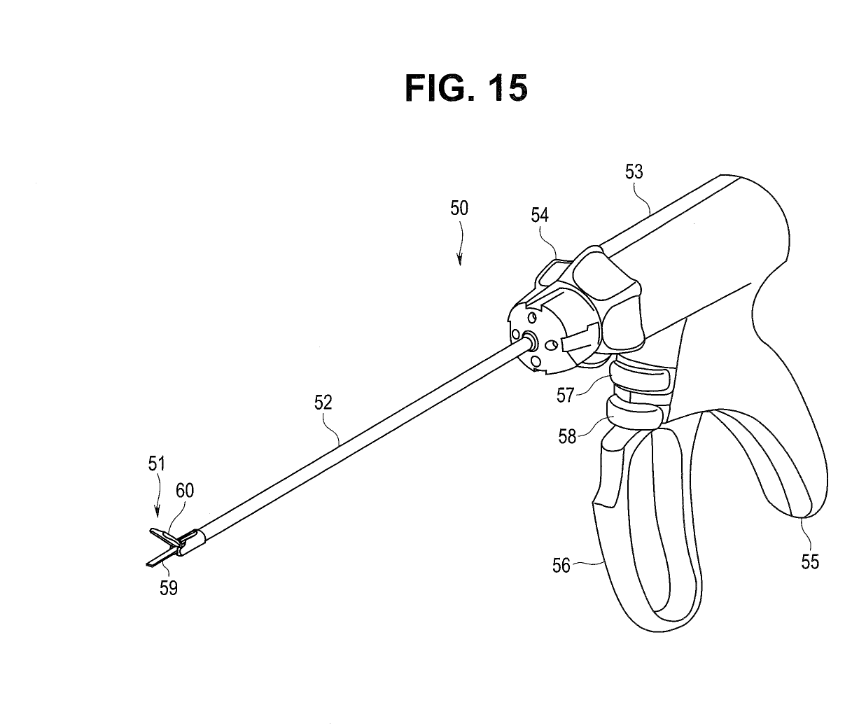

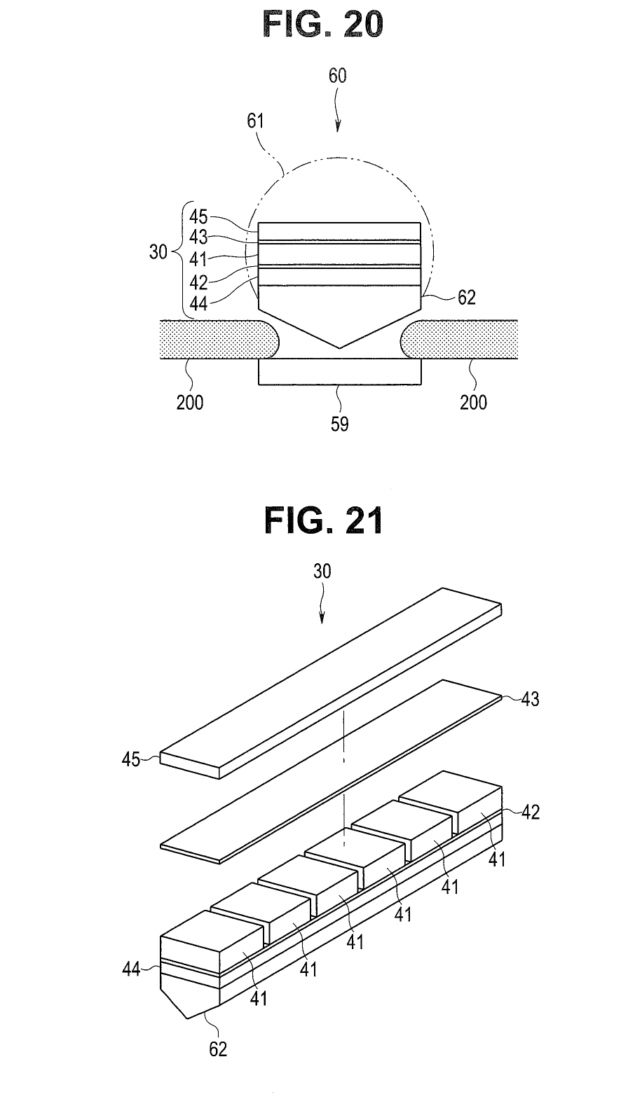

A second embodiment of the present invention is explained below with reference to FIGS. 15 to 21.

Note that, in the explanation of the present embodiment, components same as the components in the first embodiment are denoted by the same reference numerals and signs and detailed explanation of the components is omitted.

As shown in FIG. 15, a surgical operation portion device 50 functioning as a surgical treatment instrument includes a probe distal end portion 51 functioning as a treatment portion, an insertion portion 52, and an operation portion 53 in order from a distal end.

The probe distal end portion 51 includes an ultrasound probe 60 functioning as an ultrasound blade and a tabular grasping portion 59 formed of metal or the like having biocompatibility. Note that the ultrasound probe 60 is provided to be openable and closable with respect to the grasping portion 59.

Furthermore, the ultrasound probe 60 and the grasping portion 59 are provided with a first electrode and a second electrode, respectively. High-frequency current is applied with the living tissue sandwiched between the first electrode of the ultrasound probe 60 and the second electrode of the grasping portion 59, to thereby coagulate/dissect the living tissue.

The operation portion 53 includes a rotating knob 54 that rotates the insertion portion 52, a fixed handle 55 to be held during use, a movable handle 56 for opening and closing the ultrasound probe 60, a first handle switch 57 for operating ultrasound and a high-frequency output for performing coagulation/dissection of a living tissue or hemostasis/dissection of a blood vessel, and a second handle switch 58 for operating a high-frequency output for performing coagulation of the living tissue or hemostasis of the blood vessel.

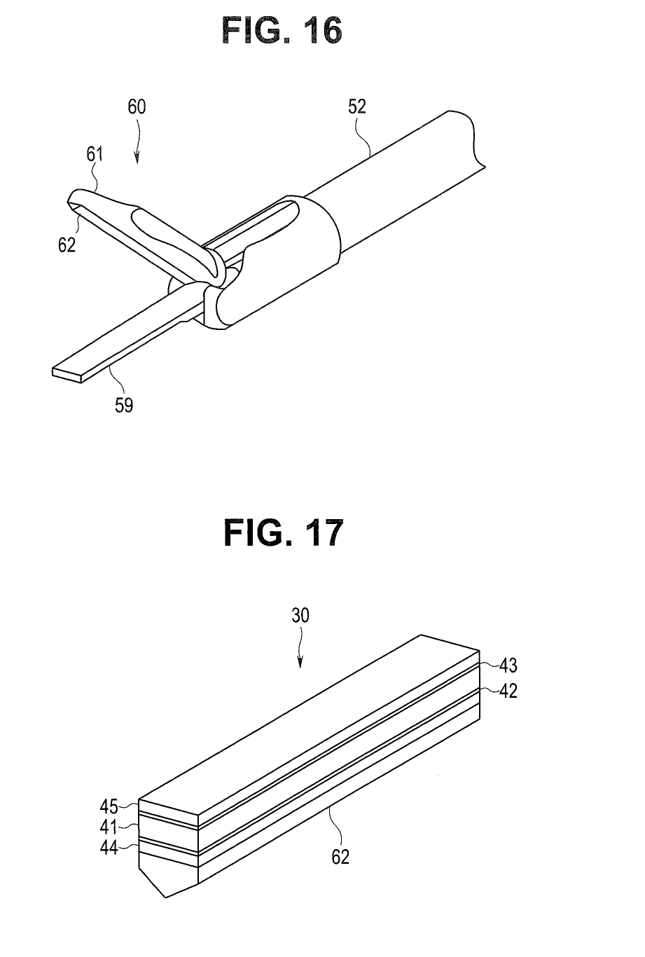

As shown in FIG. 16, the ultrasound probe 60 includes a cover body 61 that covers an upper part and a blade 62 having a pentagonal sectional shape exposed from a lower end of the cover body 61. The piezoelectric element unit 30 shown in FIG. 17 is incorporated in the cover body 61.

As shown in FIG. 17, the blade 62 is disposed in direct contact with the piezoelectric element unit 30. Note that the blade 62 functions as an ultrasound transmitting material that transmits ultrasound generated from the piezoelectric element unit 30. Like the grasping portion metal 26, the blade 62 is formed of duralumin, a titanium alloy, pure titanium, ceramics, or the like.

In the present embodiment, a shape of the blade 62 only has to be a polygon formed by, for example, a pentagonal cross section and including at least a portion of an acute angle in the cross section. The blade 62 is provided such that the cross section acute side thereof is opposed to the grasping portion 59.

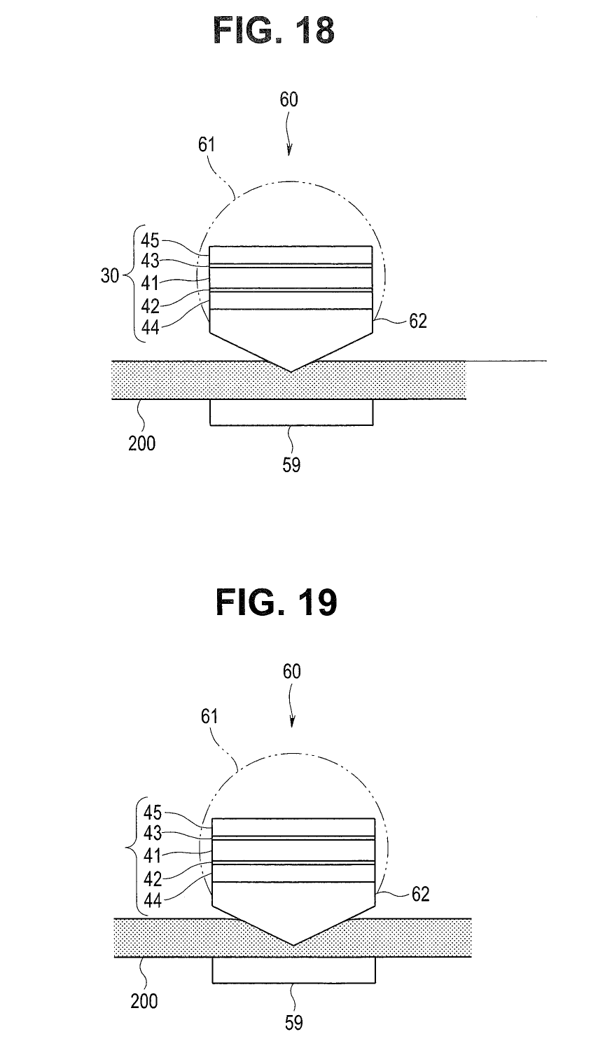

In the piezoelectric element unit 30, the insulating plate 44 is directly bonded to an upper surface of the blade 62 by a brazing material. Note that, as a metal-based bonding material used in the brazing material, the gold tin (AuSn), the metal nano-particle bonding, the solder for high temperature, the ceramics adhesive, or the like explained above is used. As shown in FIGS. 18 to 20, the surgical operation portion device 50 according to the present embodiment configured as explained above can grasp a living tissue (a blood vessel) 200 on the grasping portion 59 with the ultrasound probe 60 and drive the piezoelectric element unit 30 to coagulate (stanch)/dissect the living tissue (the blood vessel) 200 with vibration and heat.

According to the above explanation, a surgical treatment portion system including the surgical operation portion device 50 functioning as the surgical treatment instrument according to the present invention and the external apparatus is configured more simply by directly bonding the piezoelectric element unit 30 to the upper surface, which is one surface, of the blade 62. As in the first embodiment, the surgical treatment portion system can adopt a configuration with high operability that can facilitate approach to a treatment portion part or a diseased part to make it easy to grasp the living tissue and can quickly and easily apply appropriate coagulation treatment portion or dissection treatment portion to the living tissue.

### (Modification)

Note that, in the piezoelectric element unit 30 in the present embodiment, as in the first embodiment, the lithium niobate single crystal (LiNbO3) configuring the rectangular piezoelectric body 41 is likely to be cracked because thermal stress due to a difference in a coefficient of thermal expansion between members occurs during manufacturing and during driving thereof. Therefore, as shown in FIG. 21, the rectangular piezoelectric body 41 may be divided into a plurality of pieces, six pieces here, and the divided rectangular piezoelectric bodies 41 may be provided in parallel to one another to reduce a size of the individual rectangular piezoelectric bodies 41 to reduce stress applied to the rectangular piezoelectric bodies 41 during manufacturing, during operation portion, and the like and prevent the rectangular piezoelectric bodies 41 from being easily cracked.

### Third Embodiment

A third embodiment of the present invention is explained below with reference to the drawings.

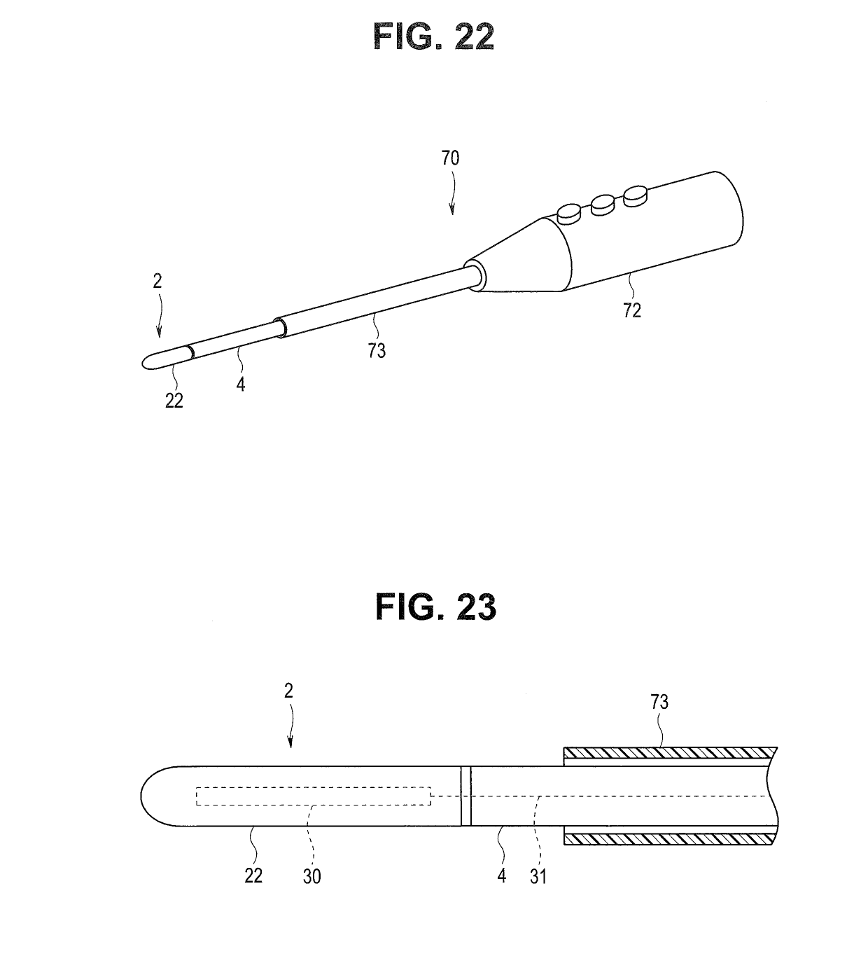

FIG. 22 is a perspective view showing a configuration of a hand piece functioning as a surgical treatment instrument. FIG. 23 is a partial sectional view showing a configuration of a distal end portion of the hand piece.

Note that, in the explanation of the present embodiment, components same as the components in the first embodiment and the second embodiment are denoted by the same reference numerals and signs and detailed explanation of the components is omitted.

As shown in FIGS. 22 and 23, a hand piece 70 functioning as an ultrasound knife used as the surgical treatment instrument to perform treatment portion such as resection of a treatment portion target living tissue includes the treatment portion 2 in which the piezoelectric element unit 30 is provided in the ultrasound probe 22 as in the first embodiment.

Note that the ultrasound probe 22 provided in the treatment portion 2 is connected to the insertion tube 4 inserted through an insulation pipe 73 extended from a grasping portion 72.

In the hand piece 70 configured as explained above, since the piezoelectric element unit 30 is mounted on the treatment portion 2, an ultrasound transducer incorporated in the grasping portion 72 as in the conventional configuration is unnecessary. Consequently, it is possible to reduce the grasping portion 72 in size and weight.

The invention described in the embodiments is not limited to the embodiments and the modifications and can be variously modified and implemented without departing from the spirit of the invention in an implementation stage. Further, the embodiments include inventions in various stages. Various inventions can be extracted by appropriate combinations in a disclosed plurality of constituent elements.

For example, when the described problems can be solved and the described effects can be obtained even if several constituent elements are deleted from all the constituent elements described in the embodiments, a configuration from which the constituent elements are deleted can be extracted as an invention.

What is claimed is:

1. A surgical treatment instrument comprising:
an insertion portion extending along a longitudinal axis;
an operation portion provided on one end side of the insertion portion along the longitudinal axis; and
a treatment portion provided on another end side of the insertion portion along the longitudinal axis, wherein the treatment portion comprises:
a piezoelectric element configured to generate ultrasound to treat a living tissue; and
an ultrasound transmitting material configured to transmit the ultrasound generated by the piezoelectric element to the living tissue.

2. The surgical treatment instrument according to claim 1, wherein the ultrasound transmitting material is provided with respect to the piezoelectric element in at least one of a first direction perpendicular to the longitudinal axis and a second direction in an opposite direction of the first direction.

3. The surgical treatment instrument according to claim 2, wherein
when the ultrasound transmitting material is provided in the first direction perpendicular to the longitudinal axis and the second direction in the opposite direction of the first direction, the ultrasound transmitting material is provided as a front mass formed on a side of the first direction and a back mass formed on a side of the second direction, and
wherein the piezoelectric element is disposed between the front mass and the back mass.

4. The surgical treatment instrument according to claim 3, wherein
the piezoelectric element is surrounded by the front mass and the back mass, and
wherein when a direction perpendicular to the first and second directions and to a direction of the longitudinal axis is represented as a width direction, at least one of the front mass and the back mass is separated from the piezoelectric element in the width direction.

5. The surgical treatment instrument according to claim 4, further comprising a filler provided between at least one of the front mass and the back mass and the piezoelectric element in the width direction.

6. The surgical treatment instrument according to claim 1, wherein the piezoelectric element is configured to generate heat for treating the living tissue.

7. The surgical treatment instrument according to claim 1, wherein the piezoelectric element is bonded to the ultrasound transmitting material using at least one of a metal-based adhesive and an inorganic adhesive.

8. The surgical treatment instrument according to claim 7, wherein a bonding material for bonding the piezoelectric element to the ultrasound transmitting material has a melting point equal to or higher than 200° C.

9. The surgical treatment instrument according to claim 1, wherein the piezoelectric element comprises a piezoelectric single crystal.

10. A surgical treatment instrument apparatus comprising:
the surgical treatment instrument according to claim 1;
a driving-signal generating circuit configured to output a driving signal to the piezoelectric element; and
a controller configured to select one of a first mode for controlling a frequency of the driving signal based on a first resonance frequency of the piezoelectric element and a second mode for controlling the frequency of the driving signal based on a second resonance frequency of the treatment portion.

11. The surgical treatment instrument apparatus according to claim 10, wherein the controller selects one of the first mode for controlling a frequency in a MHz band, which is the first resonance frequency, and the second mode for controlling a frequency in a kHz band, which is the second resonance frequency.
